SEQUENCE LISTING (41 sequences)

SEQ ID NO: 1 - DNA, 50 bp, Artificial Sequence
  Synthetic
tgacccttgagcaacacaggcttgaacttatatggggattttcttccatc

SEQ ID NO: 2 - DNA, 50 bp, Artificial Sequence
  Synthetic
tcttcagagaaatagaatcactagggaaccaaatatatatacatacaatt

SEQ ID NO: 3 - DNA, 50 bp, Artificial Sequence
  Synthetic
caggcaaggccaagccatttctgtttccaaatccactggctccctcccac

SEQ ID NO: 4 - DNA, 50 bp, Artificial Sequence
  Synthetic
tacctagcatggtacctgcaggtggcccataatcatgagttattcagtaa

SEQ ID NO: 5 - DNA, 50 bp, Artificial Sequence
  Synthetic
cttagctccaatttaaaagattaatcataaacatttgggaaggagagtga

SEQ ID NO: 6 - DNA, 50 bp, Artificial Sequence
  Synthetic
taacaaaaggctgtaacaagggctacaggaatcatgagccaggaactgtg

SEQ ID NO: 7 - DNA, 50 bp, Artificial Sequence
  Synthetic
gctctgtcacccagattggactgcagtgggggaatcatagctcactacag

SEQ ID NO: 8 - DNA, 50 bp, Artificial Sequence
  Synthetic
gtgtattccctgtgcctttgggggcatctcttatactcatgaaatcaaca

SEQ ID NO: 9 - DNA, 50 bp, Artificial Sequence
  Synthetic
taccaaagaggaaaatcacccttgtcacatacttgctattaaaatatact

SEQ ID NO: 10 - DNA, 50 bp, Artificial Sequence
  Synthetic
caagtgattccaatcatagccacagtttacaacatttgtatctttatctg

SEQ ID NO: 11 - DNA, 50 bp, Artificial Sequence
  Synthetic
tatgacaagtgttctatcataccctttatatatattaaccttaaaataac

SEQ ID NO: 12 - DNA, 50 bp, Artificial Sequence
  Synthetic
caaatattggtaattaaatgtttactatagactatttagtgagattaaaa

SEQ ID NO: 13 - DNA, 50 bp, Artificial Sequence
  Synthetic
tctacaggataggtaaataaattaaggcatattcacgcaatgggatacga

SEQ ID NO: 14 - DNA, 50 bp, Artificial Sequence
  Synthetic
ctttctgcccacacggcctggcaacttatatgtatttttgtatttcatgt

SEQ ID NO: 15 - DNA, 50 bp, Artificial Sequence
  Synthetic
agagttgttcctttaataacaagacaagggaaaaagagaactgtcagaat

SEQ ID NO: 16 - DNA, 50 bp, Artificial Sequence
  Synthetic
acctgagcattagccccaggaccaatctggtcacaaacatattaatgaat

SEQ ID NO: 17 - DNA, 50 bp, Artificial Sequence
  Synthetic
cagacagacagatgagaggggatttattagaggaattagctcaagtgata

SEQ ID NO: 18 - DNA, 50 bp, Artificial Sequence
  Synthetic
gatgaaaaaagagactgtattagtaaggcttctccagagagaaagaatca

SEQ ID NO: 19 - DNA, 50 bp, Artificial Sequence
  Synthetic
tctttttgggctgcctatggctcaacccaagttgaaggaggagatttgac

SEQ ID NO: 20 - DNA, 50 bp, Artificial Sequence
  Synthetic
tctttagtgggcatccgtgactctctggactctgacccatctaacgccta

SEQ ID NO: 21 - DNA, 50 bp, Artificial Sequence
  Synthetic
agatggatgatagatacatgcttacagatgcacacacaaacgctaaatgg

SEQ ID NO: 22 - DNA, 50 bp, Artificial Sequence
  Synthetic
tgggagcaaacaaaggcagatcccaagctcttcctcttccctagatcaat

SEQ ID NO: 23 - DNA, 50 bp, Artificial Sequence
  Synthetic
tagtagcaactgttattgtaagacatctccacacaccagagaagttaatt

SEQ ID NO: 24 - DNA, 50 bp, Artificial Sequence
  Synthetic
tgcagtgagccatgttcatgccactgcacttcactctgagtgacaaattg

SEQ ID NO: 25 - DNA, 50 bp, Artificial Sequence
  Synthetic
cctaccttctttccttcaacagaatcttattctgttgcccaggctggagt

SEQ ID NO: 26 - DNA, 50 bp, Artificial Sequence
  Synthetic
cttttctcagtctccataaatatgtgagtcaattccccaagtgaattgcc

SEQ ID NO: 27 - DNA, 50 bp, Artificial Sequence
  Synthetic
acattgactaatacaacatctttaatatatcacagtttaatttcaagtta

SEQ ID NO: 28 - DNA, 50 bp, Artificial Sequence
  Synthetic
tattgccaatcatacattcgcgtgatcactcacactgtgccgggcactct

SEQ ID NO: 29 - DNA, 50 bp, Artificial Sequence
  Synthetic
ctatagaccctgtcctagccttcttatagctgctatgggggctagatttt

SEQ ID NO: 30 - DNA, 50 bp, Artificial Sequence
  Synthetic
acctatccctctatggcaattgcttgcaaccagggagattttattcccag

SEQ ID NO: 31 - DNA, 50 bp, Artificial Sequence
  Synthetic
taacctatcatccatccttatctcttgtgtatctattcattcaatcatac

SEQ ID NO: 32 - DNA, 50 bp, Artificial Sequence
  Synthetic
cagttactgttaatatcttcattttacaggtaggaaaactgagacacagg

SEQ ID NO: 33 - DNA, 50 bp, Artificial Sequence
  Synthetic
agatactatctcctggtgcacacttggacagcatttcctgtgtcagaccc

SEQ ID NO: 34 - DNA, 50 bp, Artificial Sequence
  Synthetic
caatgggaatcaccccagagcccagataccctttgaattttgccccctat

SEQ ID NO: 35 - DNA, 50 bp, Artificial Sequence
  Synthetic
cagcctggcccacacagtcccctgtacacagggcttccgagtgcaggtca

SEQ ID NO: 36 - DNA, 50 bp, Artificial Sequence
  Synthetic
aggacagaagggcctagcgggaagggaacaggagtaagaccagcgcacag

SEQ ID NO: 37 - DNA, 50 bp, Artificial Sequence
  Synthetic
tttcagggctgtgatcactagcacccagaaccgtcgactggcacagaaca

SEQ ID NO: 38 - DNA, 50 bp, Artificial Sequence
  Synthetic
tcctaccaccagcttcccagtttaagctctgatggttggcctcaagcctg

SEQ ID NO: 39 - DNA, 50 bp, Artificial Sequence
  Synthetic
gctaccacctcatcctgggcaccctggttatatcaacttcagctatgagg

SEQ ID NO: 40 - DNA, 50 bp, Artificial Sequence
  Synthetic
tttcagcattggacaaagtacatggattacagtcaatcaaggctaactga

SEQ ID NO: 41 - DNA, 50 bp, Artificial Sequence
  Synthetic
tctgcttctcagatcctctgacactcggttgtaggtattatcacggtctg

(19) United States
(12) Patent Application Publication
(10) Pub. No.: US 2020/0005894 A1
(43) Pub. Date: Jan. 2, 2020

(54) METHOD AND SYSTEM FOR SAMPLE IDENTITY ASSURANCE

(71) Applicant: RADY CHILDREN'S HOSPITAL RESEARCH CENTER, San Diego, CA (US)

(72) Inventors: Yan Ding, San Diego, CA (US); Sergey Batalov, San Diego, CA (US)

(21) Appl. No.: 16/457,215

(22) Filed: Jun. 28, 2019

Related U.S. Application Data
(60) Provisional application No. 62/692,366, filed on Jun. 29, 2018.

(51) Int. Cl. G16B 30/00; C12Q 1/6869

(52) U.S. Cl. CPC G16B 30/00; C12Q 1/6869

(57) ABSTRACT

The present disclosure provides a method for genetic analysis including allelotyping as well as a system for implementing such analysis.

### CROSS-REFERENCE TO RELATED APPLICATIONS

This application claims benefit of priority under 35 U.S.C. § 119(e) of U.S. Ser. No. 62/692,366, filed Jun. 29, 2018, the entire contents of which is incorporated herein by reference in its entirety.

### INCORPORATION OF SEQUENCE LISTING

The material in the accompanying sequence listing is hereby incorporated by reference into this application. The accompanying sequence listing text file, name RADY_1_Sequence_Listing.txt, was created on Jun. 25, 2019, and is 8 kb. The file can be accessed using Microsoft Word on a computer that uses Windows OS.

### BACKGROUND OF THE INVENTION

### Field of the Invention

The invention relates generally to genetic analysis and more specifically to a method and system for allelotyping to ensure sample identity.

### Background Information

Whole Genome Sequencing (WGS), Whole Exome Sequencing (WES) and Targeted Gene Panel Sequencing using the Next Generation Sequencing (NGS) platforms are complicated processes involving multiple procedural steps. Sample swap or contamination during the process in NGS may result in false positive variant detections and genotype misclassification. The assurance of sample identity throughout the process is a critical quality control component. The process to ensure correct sample identity is a challenge for sequencing facilities.

Currently, some NGS facilities are performing array-based genotyping and using single nucleotide polymorphism (SNP) to obtain the concordance between genotype profiling called from NGS data and that from array-based genotype data (SNP microarrays). It is known that errors related or unrelated to specific processes may occur in array-based genotyping and lead to disconcordant genotype calls between SNP array data and NGS data. Meanwhile, the depth coverage of NGS impacts SNP calls from NGS data especially for lower minor allele frequencies (MAF) SNPs. For NGS panel sequencing, a custom designed array workflow has to be created to optimize concordance between NGS panel data and SNP microarray data. The work flow requires 2-3 days to complete. Additionally, for laboratories with relatively small sample volumes, initial instrumentation, modifications of cost and staffing models may need to be developed.

Improved methods for assuring correct sample identity are needed when performing genetic analysis.

### SUMMARY OF THE INVENTION

The present invention provides a method and system for conducting genetic analysis via allelotyping. The method utilizes a combination of different types of allelotyping techniques to ensure correct sample identity.

Accordingly, in one aspect, the invention provides a method for performing genetic analysis. The method includes:

a) determining a first allelotype for a sample via short tandem repeat (STR) amplification;

b) determining a second allelotype for the sample via genetic sequencing; and

c) determining allele concordance between the first allelotype and the second allelotype.

In embodiments, the method further includes generating an allele profiling concordance table. In one embodiment, the method includes calculating a statistical probability to determine whether the first allelotype and the second allelotype are of a single subject.

In various embodiments, genetic sequencing includes whole genome sequencing (WGS) or rapid whole genome sequencing (rWGS) or whole exome sequencing (WES), next-generation sequence (NGS), targeted gene panel sequencing, or a combination thereof.

In embodiments where sequencing includes WES or targeted gene panel sequencing, a panel having one or more oligonucleotides selected from SEQ ID NOs: 1-41 is utilized which enables allelotyping in these applications.

Accordingly, the invention further provides a panel having one or more oligonucleotides selected from SEQ ID NOs: 1-41. In embodiments, each oligonucleotide is between about 50 to 120 nucleotides in length. In one embodiment, each oligonucleotide is between about 50 nucleotides in length or greater. In one embodiment, each oligonucleotide is 120 nucleotides in length or less.

In an embodiment the invention provides a genetic analysis system configured to perform a method of the disclosure. The system includes: a) at least one processor operatively connected to a memory; b) a receiver component configured to receive DNA analysis information including sequence information generated from PCR amplification of DNA in a DNA sample; and c) an analysis component, executed by the at least one processor, configured to perform a method of the disclosure, such as determining an allelotype, generating an allele profiling concordance table and calculating a statistical probability to determine whether a first allelotype and a second allelotype are of a single subject.

In another embodiment, the invention provides a system for performing the method of the invention. The system includes a controller having at least one processor and non-transitory memory. The controller is configured to perform one or more of the processes of the method as described herein.

In still another embodiment, the invention provides a non-transitory computer readable storage medium encoded with a computer program. The program includes instructions that, when executed by one or more processors, cause the one or more processors to perform operations that implement a method of the disclosure.

In yet another embodiment, the invention provides a computing system. The system includes a memory, and one or more processors coupled to the memory, with the one or more processors being configured to perform operations that implement a method of the disclosure.

### DETAILED DESCRIPTION OF THE INVENTION

The present invention is based on an innovative method for ensuring sample identity which includes a combination of multiple allelotyping techniques. The presently disclosed methodology includes comparing the concordance of STR (Short Tandem Repeat) allele profiling generated by the GlobalFiler™ PCR Amplification kit and by NGS using LobSTR™ software to assure sample identity and to detect potential cross contamination among the different samples.

GlobalFiler™ panel allows the determination of allelic states of 24 positions in the human genome, as well as to identify an event of contamination (mix) of more than one sample. Computational workflow on the WGS or WES or NGS Panel (in which the SEQ ID NOs: 1-41 oligonucleotides have been included in pool down probe design) data set using an in silico STR inference software (such as lobSTR™) allows the independent determination of allelic states of the same 24 positions in human genome. Statistical framework allows one to rule out any reasonable doubt (the probability of error less than 1/1,000,000,000,000,000) that the two samples came from the same individual if no less than 18 out of 24 positions match.

Concordance between allelotype profiling called by STR and by WGS or WES is high and consistent. STR genotyping using GlobalFiler™ can generate consistent loci profiling with high accuracy and sensitivity. The work flow is simpler and easier for laboratory technologist to complete within 4-6 hours. Setting up STR reactions does not require as large a batching set as microarray. Additionally, reagents are not lost with a smaller sample set in a batch.

Before the present compositions and methods are described, it is to be understood that this invention is not limited to particular methods and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

Methods

The present invention provides a method for conducting genetic analysis via allelotyping. The method utilizes a combination of different types of allelotyping techniques to ensure sample identity.

Accordingly, in one aspect, the invention provides a method for performing genetic analysis. The method includes:

a) determining a first allelotype for a sample via short tandem repeat (STR) amplification;

b) determining a second allelotype for the sample via genetic sequencing; and

c) determining allele concordance between the first allelotype and the second allelotype.

The method of the disclosure contemplates genetic sequencing to generate an allelotype.

Sequencing may be by any method known in the art. Sequencing methods include, but are not limited to, Maxam-Gilbert sequencing-based techniques, chain-termination-based techniques, shotgun sequencing, bridge PCR sequencing, single-molecule real-time sequencing, ion semiconductor sequencing (Ion Torrent™ sequencing), nanopore sequencing, pyrosequencing (454), sequencing by synthesis, sequencing by ligation (SOLiD™ sequencing), sequencing by electron microscopy, dideoxy sequencing reactions (Sanger method), massively parallel sequencing, polony sequencing, and DNA nanoball sequencing. In some embodiments, sequencing involves hybridizing a primer to the template to form a template/primer duplex, contacting the duplex with a polymerase enzyme in the presence of a detectably labeled nucleotides under conditions that permit the polymerase to add nucleotides to the primer in a template-dependent manner, detecting a signal from the incorporated labeled nucleotide, and sequentially repeating the contacting and detecting steps at least once, wherein sequential detection of incorporated labeled nucleotide determines the sequence of the nucleic acid. In some embodiments, the sequencing comprises obtaining paired end reads.

In some embodiments, sequencing of nucleic acid is performed using whole genome sequencing (WGS), rapid WGS, whole exome sequencing (WES), targeted gene panel sequencing, next-generation sequencing (NGS), or any combination thereof. In some embodiments, targeted sequencing is performed and may be either DNA or RNA sequencing. The targeted sequencing may be to a subset of the whole genome. In some embodiments the targeted sequencing is to introns, exons, non-coding sequences or a combination thereof. The DNA is sequenced using a NGS platform, which is massively parallel sequencing. NGS technologies provide high throughput sequence information, and provide digital quantitative information, in that each sequence read that aligns to the sequence of interest is countable. In certain embodiments, clonally amplified DNA templates or single DNA molecules are sequenced in a massively parallel fashion within a flow cell (e.g., as described in WO 2014/015084). In addition to high-throughput sequence information, NGS provides quantitative information, in that each sequence read is countable and represents an individual clonal DNA template or a single DNA molecule. The sequencing technologies of NGS include pyro sequencing, sequencing-by-synthesis with reversible dye terminators, sequencing by oligonucleotide probe ligation and ion semiconductor sequencing. DNA from individual samples can be sequenced individually (i.e., singleplex sequencing) or DNA from multiple samples can be pooled and sequenced as indexed genomic molecules (i.e., multiplex sequencing) on a single sequencing run, to generate up to several hundred million reads of DNA sequences. Commercially available platforms include, e.g., platforms for sequencing-by-synthesis, ion semiconductor sequencing, pyrosequencing, reversible dye terminator sequencing, sequencing by ligation, single-molecule sequencing, sequencing by hybridization, and nanopore sequencing. In embodiments, the methodology of the disclosure utilizes systems such as those provided by Illumina, Inc, (HiSeq™ X10, HiSeq™ 1000, HiSeq 2000, HiSeq™ 2500, HiSeq™ 4000, NovaSeq 5000, NovaSeq™ 6000, Genome Analyzers™, MiSeq™ systems), Applied Biosystems Life Technologies (ABI PRISM™ Sequence detection systems, SOLiD™ System, Ion PGM™ Sequencer, ion Proton™ Sequencer).

The terms “polynucleotide,” “nucleotide sequence,” “nucleic acid,” and “oligonucleotide” are used interchangeably. They refer to a polymeric form of nucleotides of any length, either deoxyribonucleotides or ribonucleotides, or analogs thereof. Polynucleotides may have any three-dimensional structure, and may perform any function, known or unknown. Polynucleotides may be single- or multi-stranded (e.g., single-stranded, double-stranded, and triple-helical) and contain deoxyribonucleotides, ribonucleotides, and/or analogs or modified forms of deoxyribonucleotides or ribonucleotides, including modified nucleotides or bases or their analogs. Because the genetic code is degenerate, more than one codon may be used to encode a particular amino acid, and the present invention encompasses polynucleotides which encode a particular amino acid sequence. Any type of modified nucleotide or nucleotide analog may be used, so long as the polynucleotide retains the desired functionality under conditions of use, including modifications that increase nuclease resistance (e.g., deoxy, 2′-O-Me, phosphorothioates, and the like). Labels may also be incorporated for purposes of detection or capture, for example, radioactive or nonradioactive labels or anchors, e.g., biotin. The term polynucleotide also includes peptide nucleic acids (PNA). Polynucleotides may be naturally occurring or non-naturally occurring. Polynucleotides may contain RNA, DNA, or both, and/or modified forms and/or analogs thereof. A sequence of nucleotides may be interrupted by non-nucleotide components. One or more phosphodiester linkages may be replaced by alternative linking groups. These alternative linking groups include, but are not limited to, embodiments wherein phosphate is replaced by P(O)S (“thioate”), P(S)S (“dithioate”), (O)NR₂ (“amidate”), P(O)R, P(O)OR′, CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. The following are non-limiting examples of polynucleotides: coding or non-coding regions of a gene or gene fragment, intergenic DNA, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), small nucleolar RNA, ribozymes, cDNA, recombinant polynucleotides, branched polynucleotides, plasmids, vectors, isolated DNA of any sequence, isolated RNA of any sequence, nucleic acid probes, adapters, and primers. A polynucleotide may include modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component, tag, reactive moiety, or binding partner. Polynucleotide sequences, when provided, are listed in the 5′ to 3′ direction, unless stated otherwise.

In embodiments, sequencing includes use of a panel of oligonucleotides. For example, a panel is useful where sequencing includes WES or targeted gene panel sequencing.

As such, the invention provides a panel having one or more oligonucleotides. In embodiments, the oligonucleotides include one or more oligonucleotides selected from SEQ ID NOs: 1-41 as shown in Table I.

TABLE I

| | | SEQ |
|---|---|---|
| Oligonucleotide | | ID |
| Probe Name | Sequence | NO |
| D1S1656_D | TGACCCTTGAGCAACACAGGCTTGAACTTATATGGGGATTTTCTTCCATC | 1 |
| D1S1656_U | TCTTCAGAGAAATAGAATCACTAGGGAACCAAATATATATACATACAATT | 2 |
| D2S1338_D | CAGGCAAGGCCAAGCCATTTCTGTTTCCAAATCCACTGGCTCCCTCCCAC | 3 |
| D2S1338_U | TACCTAGCATGGTACCTGCAGGTGGCCCATAATCATGAGTTATTCAGTAA | 4 |
| D2S441_D | CTTAGCTCCAATTTAAAAGATTAATCATAAACATTTGGGAAGGAGAGTGA | 5 |
| D2S441_U | TAACAAAAGGCTGTAACAAGGGCTACAGGAATCATGAGCCAGGAACTGTG | 6 |
| D3S1358_D | GCTCTGTCACCCAGATTGGACTGCAGTGGGGGAATCATAGCTCACTACAG | 7 |
| D3S1358_U | GTGTATTCCCTGTGCCTTTGGGGGCATCTCTTATACTCATGAAATCAACA | 8 |
| D5S818_D | TACCAAAGAGGAAAATCACCCTTGTCACATACTTGCTATTAAAATATACT | 9 |
| D5S818_U | CAAGTGATTCCAATCATAGCCACAGTTTACAACATTTGTATCTTTATCTG | 10 |
| D7S820_D | TATGACAAGTGTTCTATCATACCCTTTATATATATTAACCTTAAAATAAC | 11 |
| D7S820_U | CAAATATTGGTAATTAAATGTTTACTATAGACTATTTAGTGAGATTAAAA | 12 |
| D8S1179_D | TCTACAGGATAGGTAAATAAATTAAGGCATATTCACGCAATGGGATACGA | 13 |
| D8S1179_U | CTTTCTGCCCACACGGCCTGGCAACTTATATGTATTTTTGTATTTCATGT | 14 |
| D10S1248_D | AGAGTTGTTCCTTTAATAACAAGACAAGGGAAAAAGAGAACTGTCAGAAT | 15 |
| D10S1248_U | ACCTGAGCATTAGCCCCAGGACCAATCTGGTCACAAACATATTAATGAAT | 16 |
| D12S391_D | CAGACAGACAGATGAGAGGGGATTTATTAGAGGAATTAGCTCAAGTGATA | 17 |
| D12S391_U | GATGAAAAAAGAGACTGTATTAGTAAGGCTTCTCCAGAGAGAAAGAATCA | 18 |
| D13S317_D | TCTTTTTGGGCTGCCTATGGCTCAACCCAAGTTGAAGGAGGAGATTTGAC | 19 |
| D13S317_U | TCTTTAGTGGGCATCCGTGACTCTCTGGACTCTGACCCATCTAACGCCTA | 20 |
| D16S539_D | AGATGGATGATAGATACATGCTTACAGATGCACACACAAACGCTAAATGG | 21 |
| D16S539_U | TGGGAGCAAACAAAGGCAGATCCCAAGCTCTTCCTCTTCCCTAGATCAAT | 22 |
| D18S51_D | TAGTAGCAACTGTTATTGTAAGACATCTCCACACACCAGAGAAGTTAATT | 23 |
| D18S51_U | TGCAGTGAGCCATGTTCATGCCACTGCACTTCACTCTGAGTGACAAATTG | 24 |
| D19S433_D | CCTACCTTCTTTCCTTCAACAGAATCTTATTCTGTTGCCCAGGCTGGAGT | 25 |
| D21S11_D | CTTTTCTCAGTCTCCATAAATATGTGAGTCAATTCCCCAAGTGAATTGCC | 26 |
| D21S11_U | ACATTGACTAATACAACATCTTTAATATATCACAGTTTAATTTCAAGTTA | 27 |
| D22S1045_D | TATTGCCAATCATACATTCGCGTGATCACTCACACTGTGCCGGGCACTCT | 28 |
| D22S1045_U | CTATAGACCCTGTCCTAGCCTTCTTATAGCTGCTATGGGGGCTAGATTTT | 29 |
| DYS391_D | ACCTATCCCTCTATGGCAATTGCTTGCAACCAGGGAGATTTTATTCCCAG | 30 |
| DYS391_U | TAACCTATCATCCATCCTTATCTCTTGTGTATCTATTCATTCAATCATAC | 31 |
| CSF1PO_D | CAGTTACTGTTAATATCTTCATTTTACAGGTAGGAAAACTGAGACACAGG | 32 |
| CSF1PO_U | AGATACTATCTCCTGGTGCACACTTGGACAGCATTTCCTGTGTCAGACCC | 33 |
| TH01_D | CAATGGGAATCACCCCAGAGCCCAGATACCCTTTGAATTTTGCCCCCTAT | 34 |
| TH01_U | CAGCCTGGCCCACACAGTCCCCTGTACACAGGGCTTCCGAGTGCAGGTCA | 35 |
| TPOX_D | AGGACAGAAGGGCCTAGCGGGAAGGGAACAGGAGTAAGACCAGCGCACAG | 36 |
| TPOX_U | TTTCAGGGCTGTGATCACTAGCACCCAGAACCGTCGACTGGCACAGAACA | 37 |
| AMEL_D | TCCTACCACCAGCTTCCCAGTTTAAGCTCTGATGGTTGGCCTCAAGCCTG | 38 |
| AMEL_U | GCTACCACCTCATCCTGGGCACCCTGGTTATATCAACTTCAGCTATGAGG | 39 |
| YIND | TTTCAGCATTGGACAAAGTACATGGATTACAGTCAATCAAGGCTAACTGA | 40 |
| FGA | TCTGCTTCTCAGATCCTCTGACACTCGGTTGTAGGTATTATCACGGTCTG | 41 |

Polynucleotides of the present invention, such as oligonucleotides of the panel of the invention may be DNA or RNA molecules of any suitable length. For example, one of skill in the art would understand what lengths are suitable for oligonucleotides to be utilized in targeted gene panels. Such molecules are typically from about 50 to 150, 50 to 140, 50 to 130, 50 to 120, 50 to 110, 50 to 100, 50 to 100, 50 to 90, 50 to 80, 50 to 70 or 50 to 60 nucleotides in length. For example the molecule may be about 50, 55, 60, 65, 70, 75, 80, 85, 90, 95, 100, 105, 110, 115 or 120 nucleotides in length. Such polynucleotides may include from at least about 50 to about 120 nucleotides or more, including at least about 50 nucleotides, at least about 55 nucleotides, at least about 60 nucleotides, at least about 65 nucleotides, at least about 70 nucleotides, at least about 75 nucleotides, at least about 80 nucleotides, at least about 85 nucleotides, at least about 90 nucleotides, at least about 95 nucleotides, at least about 100 nucleotides, at least about 110 nucleotides, at least about 120 nucleotides or greater than 120 nucleotides.

As used herein, “polypeptide” refers to a composition comprised of amino acids and recognized as a protein by those of skill in the art. The conventional one-letter or three-letter code for amino acid residues is used herein. The terms “polypeptide” and “protein” are used interchangeably herein to refer to polymers of amino acids of any length. The polymer may be linear or branched, it may include modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, synthetic amino acids and the like), as well as other modifications known in the art.

As used herein, the term “sample” herein refers to any substance containing or presumed to contain nucleic acid. The sample can be a biological sample obtained from a subject. The nucleic acids can be RNA, DNA, e.g., genomic DNA, mitochondrial DNA, viral DNA, synthetic DNA, or cDNA reverse transcribed from RNA. The nucleic acids in a nucleic acid sample generally serve as templates for extension of a hybridized primer. In some embodiments, the biological sample is a biological fluid sample. The fluid sample can be whole blood, plasma, serum, ascites, cerebrospinal fluid, sweat, urine, tears, saliva, buccal sample, cavity rinse, feces or organ rinse. The fluid sample can be an essentially cell-free liquid sample (e.g., plasma, serum, sweat, urine, and tears). In other embodiments, the biological sample is a solid biological sample, e.g., feces or tissue biopsy, e.g., a tumor biopsy. A sample can also comprise in vitro cell culture constituents (including but not limited to conditioned medium resulting from the growth of cells in cell culture medium, recombinant cells and cell components). In some embodiments, the sample is a biological sample that is a mixture of nucleic acids from multiple sources, i.e., there is more than one contributor to a biological sample, e.g., two or more individuals. In one embodiment the biological sample is a dried blood spot.

In the present invention, the subject is typically a human but also can be any species with methylation marks on its genome, including, but not limited to, a dog, cat, rabbit, cow, bird, rat, horse, pig, or monkey.

Computer Systems

The present invention is described partly in terms of functional components and various processing steps. Such functional components and processing steps may be realized by any number of components, operations and techniques configured to perform the specified functions and achieve the various results. For example, the present invention may employ various biological samples, biomarkers, elements, materials, computers, data sources, storage systems and media, information gathering techniques and processes, data processing criteria, statistical analyses, regression analyses and the like, which may carry out a variety of functions. In addition, although the invention is described in relation to genetic analysis, the present invention may be practiced in conjunction with any number of applications, environments and data analyses; the systems described herein are merely exemplary applications for the invention.

Methods for genetic analysis according to various aspects of the present invention may be implemented in any suitable manner, for example using a computer program operating on the computer system. An exemplary genetic analysis system, according to various aspects of the present invention, may be implemented in conjunction with a computer system, for example a conventional computer system comprising a processor and a random access memory, such as a remotely-accessible application server, network server, personal computer or workstation. The computer system also suitably includes additional memory devices or information storage systems, such as a mass storage system and a user interface, for example a conventional monitor, keyboard and tracking device. The computer system may, however, comprise any suitable computer system and associated equipment and may be configured in any suitable manner. In one embodiment, the computer system comprises a stand-alone system. In another embodiment, the computer system is part of a network of computers including a server and a database.

The software required for receiving, processing, and analyzing genetic information may be implemented in a single device or implemented in a plurality of devices. The software may be accessible via a network such that storage and processing of information takes place remotely with respect to users. The genetic analysis system according to various aspects of the present invention and its various elements provide functions and operations to facilitate genetic analysis, such as data gathering, processing and/or analysis. The present genetic analysis system maintains information relating to samples and facilitates analysis, For example, in the present embodiment, the computer system executes the computer program, which may receive, store, search, analyze, and report information relating to the genome. The computer program may comprise multiple modules performing various functions or operations, such as a processing module for processing raw data and generating supplemental data and an analysis module for analyzing raw data and supplemental data to perform genetic analysis.

The procedures performed by the genetic analysis system may comprise any suitable processes to facilitate genetic analysis. In one embodiment, the genetic analysis system is configured to determine allele concordance.

The genetic analysis system may also provide various additional modules and/or individual functions. For example, the genetic analysis system may also include a reporting function, for example to provide information relating to the processing and analysis functions. The genetic analysis system may also provide various administrative and management functions, such as controlling access and performing other administrative functions.

The following example is provided to further illustrate the advantages and features of the present invention, but it is not intended to limit the scope of the invention. While this example is typical of those that might be used, other procedures, methodologies, or techniques known to those skilled in the art may alternatively be used.

What is claimed is:

1. A method comprising:
a) determining a first allelotype for a sample via short tandem repeat (STR) amplification;
b) determining a second allelotype for the sample via genetic sequencing; and
c) determining allele concordance between the first allelotype and the second allelotype.

2. The method of claim 1, wherein (c) comprises generating an allele profiling concordance table.

3. The method of claim 1, further comprising calculating a statistical probability to determine whether the first allelotype and the second allelotype are of a single subject.

4. The method of claim 3, wherein the subject is human.

5. The method of claim 1, wherein the first allelotype is generated via GeneMapper™.

6. The method of claim 1, wherein the second allelotype is generated via lobSTR™.

7. The method of claim 1, wherein the sample is a biological sample.

8. The method of claim 1, wherein the sample is whole blood, plasma, serum, ascites, cerebrospinal fluid, sweat, urine, tears, saliva, buccal sample, cavity rinse, feces, organ rinse, hair or skin.

9. The method of claim 1, wherein the sample is blood.

10. The method of claim 1, wherein genetic sequencing comprises whole genome sequencing (WGS), rapid whole genome sequencing (rWGS), whole exome sequencing (WES), next-generation sequence (NGS), targeted gene panel sequencing, or a combination thereof.

11. The method of claim 10, wherein WES or targeted gene panel sequencing comprises a panel having one or more oligonucleotides selected from the group consisting of SEQ ID NOs: 1-41.

12. The method of claim 11, wherein each oligonucleotide is between about 50 to 120 nucleotides in length.

13. The method of claim 11, wherein each oligonucleotide is 50 nucleotides in length or greater.

14. The method of claim 11, wherein each oligonucleotide is 120 nucleotides in length or less.

15. The method of claim 1, wherein (a) and (b) are performed in parallel.

16. A panel comprising one or more oligonucleotides selected from the group consisting of SEQ ID NOs: 1-41.

17. The panel of claim 16, wherein each oligonucleotide is between about 50 to 120 nucleotides in length.

18. The panel of claim 16, wherein each oligonucleotide is 50 nucleotides in length or greater.

19. The panel of claim 16, wherein each oligonucleotide is 120 nucleotides in length or less.

20. A genetic analysis system comprising: a) at least one processor operatively connected to a memory; b) a receiver component configured to receive DNA analysis information including sequence information generated from PCR amplification of DNA in a DNA sample; and c) an analysis component, executed by the at least one processor, configured to determine: i) an allelotype from the sequence information; ii) generate an allele profiling concordance table; and iii) calculate a statistical probability to determine whether a first allelotype and a second allelotype are from a single subject.

21. A genetic analysis system comprising: a) at least one processor operatively connected to a memory; b) a receiver component configured to receive DNA analysis information including sequence information generated from PCR amplification of DNA in a DNA sample; and c) an analysis component, executed by the at least one processor, configured to perform (a)-(c) of claim 1.
